(19) United States
(12) Patent Application Publication
(10) Pub. No.: US 2007/0066982 A1
(43) Pub. Date: Mar. 22, 2007

(54) Fistulary catheter

(76) Inventors: Justin L. Regner, Little Rock, AR (US); Jacobo Nurko, Little Rock, AR (US); J. Ralph Broadwater JR., Little Rock, AR (US); Michael J. Edwards, Little Rock, AR (US); William C. Culp, Little Rock, AR (US)

Correspondence Address: WRIGHT, LINDSEY & JENNINGS LLP, 200 WEST CAPITOL AVENUE, SUITE 2300, LITTLE ROCK, AR, 72201-3699, US

(21) Appl. No.: 11/229,970

(22) Filed: Sep. 19, 2005

(51) Int. Cl. A61B 17/08

(52) U.S. Cl. 606/153

(57) ABSTRACT

A fistulary catheter for creating a sutureless mucosa-to-mucosa anastomosis. The catheter includes a rigid guide portion having a penetrating point and a fenestrated portion which has at least one fenestration communicating with a hollow interior and terminating in an opening which has an everted rim. The everted rim is desirably provided with a corrugated margin to grip the mucosa without compromising the blood supply. The guide portion may be hook shaped to guide the direction of the catheter or a metal probe may be used to guide a straight catheter. The guide portion may be flattened to minimize leakage around the guide portion. The fenestrated portion of the catheter may be straight or may be provided with varying degrees of tapering. The taper acts to hold the mucosa in place during the formation of the anastomosis. In an alternative embodiment, the fenestrated portion is straight without any taper and is used with spacers with differing diameters. The spacers are placed over the catheter so as to fit against the everted rim. Rather than employing the taper to hold the enteric mucosa in place, the thickness of the wall of the spacer defines a “lip” at its upper end to grip the mucosa.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention relates to a fistulary catheter for creating a sutureless anastomosis , and in particular, to an hepatico-enteric fistulary catheter.

2. Brief Description of the Related Art

are various medical reasons for creating an anastomosis, i.e., an opening or connection between two vessels or between two normally separate spaces within the human body. For example, it may be desirable to create a passage to allow the drainage of fluids from an organ whose normal outlet is blocked. One such condition involves the blockage of the bile ducts which requires the creation of a communication between the liver and the small intestine.

A method for repairing a traumatic stricture of the biliary ducts is described by Wexler and Smith in “Jejunal Mucosal Graft,” The American Journal of Surgery, Vol. 129, February 1975, pp. 204-211. It is stated that for a successful repair, the anastomosis requires epithelial to epithelial apposition. In this procedure, the opening to the bile duct is exposed and forceps are passed into the duct and through the liver substance to the surface of the liver. A latex rubber tube is grasped by the forceps, drawn back through the liver and out the opening of the bile duct. A Roux-en-Y procedure fashions a defunctionalized limb of the small intestine (“the Roux limb”). A disk of seromuscular tissue is removed the Roux limb, leaving the enteric muscosa to form a protruding sleeve with a small central hole. Side holes are made in the rubber tube, which is passed through the hole in the mucosal sleeve and anchored with sutures to the Roux limb. By pulling on the rubber tube, the enteric mucosal sleeve is brought into contact with the epithelium of the bile duct and anchored with sutures.

The limitations of the prior art are overcome by the present invention as described below.

### BRIEF SUMMARY OF THE INVENTION

The present invention is a fistulary catheter for creating a sutureless anastomosis. The catheter includes a rigid guide portion having a penetrating point. The guide portion may be reinforced for increased rigidity. For example, a wire may be embedded into the guide portion. The catheter also includes a fenestrated portion which has at least one fenestration communicating with a hollow interior. The number of fenestrations is not significant and may be adjusted to particular situations. The fenestrated portion terminates in an opening which has an everted rim. The everted rim is desirably provided with a corrugated margin. The corrugations grip the enteric mucosa but will not compromise the blood supply.

In order to create an hepatico-enteric sutureless anastomosis, a Roux limb is created and an enterotomy is made in the Roux limb. A mucosal sleeve is made as described by Wexler and Smith. The catheter of the present invention is inserted through the enterotomy and the guide portion is passed through the mucosal sleeve and into the opening of the bile duct of the liver. The rigid guide portion is then treaded through the bile duct. (The guide portion may be hook shaped to guide the direction of the catheter or a metal probe may be used to guide a straight catheter.) When resistance is felt, the penetrating point of the guide portion is forced through to the surface of the liver. The exposed guide portion is then pulled to create the anastomosis by bringing the enteric mucosal sleeve into apposition with the epithelium of the bile duct. The guide portion is then sutured to the liver at the surface and the exposed guide portion is cut off. If the guide portion is sutured to the liver with an absorbable suture, such as a polydioxanone suture, the catheter will self-dislodge into the enteric tract after the anastomosis is stable and intact. The end result is a permanent hepatico-enteric anastomosis for the drainage of bile into the lumen of the enteric tract.

The catheter may be made of various materials, such as latex, vinyl chloride or Silastic®. Silastic® will not promote an inflammatory reaction at the site where the guide portion exits the surface of the liver. However, an inflammatory reaction may be desirable to better seal the exit site. For this purpose, latex is the preferred material. Flattening the guide portion will also help to prevent leaking at the exit site.

The catheter may be used for various procedures where it is necessary to produce a communication between two normally separate spaces in the body. While the preferred embodiment of the present invention is directed to an hepatico-enteric anastomosis, the invention is not limited thereto, but is applicable to any procedures requiring the creation of a communication between two spaces in the body.

The fistulary catheter of the present invention minimizes the technical difficulties of the hepatico-enteric anastomosis, diminishes operative time, decreases the risk of biliary tree injury, allows multiple hepatico-enteric (HE) anastomoses in a very short time, and eliminates the need for second interventions to remove temporary tubes, catheters or stents. With a sutureless anastomosis, the present invention decreases the risk of future biliary strictures and ascending cholangitis. The catheter also provides a bridge to catheter-based anastomosis under laparoscopy.

The fenestrated portion of the catheter may be straight or may be provided with varying degrees of tapering. The taper acts to hold the enteric mucosa sleeve in place during the formation of the anastomosis. The taper also allows some accommodation to varying sizes of openings in the Roux limb.

In an alternative embodiment, the fenestrated portion is straight without any taper. This embodiment is capable of accommodating various intra-hepatic bile duct diameters with the use of spacers with differing diameters. The spacers are placed over the catheter so as to fit against the everted rim. Rather than employing the taper to hold the enteric mucosa in place, the thickness of the wall of the spacer defines a “lip” at its upper end to grip the enteric mucosa and provide good contact with the epithelium of the bile duct to ensure a good anastomosis.

### BRIEF DESCRIPTION OF THE SEVERAL VIEWS OF THE DRAWINGS

FIG. 1 is a perspective view of the catheter of the present invention.

FIG. 2 is a perspective view of an anastomosis formed using the catheter of the present invention.

FIG. 3 is a elevation view of the catheter of FIG. 1.

FIGS. 4, 5, 6, 7 and 8 are cross-sectional views of the guide portion of the catheter of FIG. 3 taken along the lines 4-4, 5-5, 6-6, 7-7, and 8-8, respectively, of FIG. 3.

FIG. 9 is a perspective view of a liver and small intestine showing the connection between the bile duct of the liver and the small intestine.

FIG. 10 is a perspective view of an initial step in performing an hepatico-enteric anastomosis using the catheter of the present invention. In this step the catheter is inserted through a mucosal sleeve made in a Roux limb of the small intestine.

FIG. 11 is a perspective view of a next step in performing an hepatico-enteric anastomosis in which the catheter is inserted into the bile duct of the liver.

FIG. 12 is a perspective view of a further step in which the catheter is pushed through to the surface of the liver and the mucosal sleeve is drawn tight against the mucosa of the bile duct.

FIG. 13 is a partial elevation cross-sectional view of the catheter of FIG. 12 taken along the line 13-13.

FIG. 14 is a partial elevation cross-sectional view of an alternative embodiment of the catheter of the present invention in which the fenestrated portion is straight rather than tapered.

FIGS. 15, 16 and 17 are perspective views of the spacer for use with the embodiment of FIG. 14. FIGS. 15, 16 and 17 show the spacer with differing diameters for accommodating differing diameters of bile ducts.

FIG. 18 is a partial elevation cross-sectional view of a further alternative embodiment of the catheter of the present invention in which the guide portion is straight rather than hook-shaped.

FIGS. 19 and 20 are cross-sectional views of the straight guide portions of the embodiment of FIG. 18 taken along the lines 19-19 and 20-20 of FIG. 18 showing an embedded reinforcement wire in the guide portion.

### DETAILED DESCRIPTION OF THE INVENTION

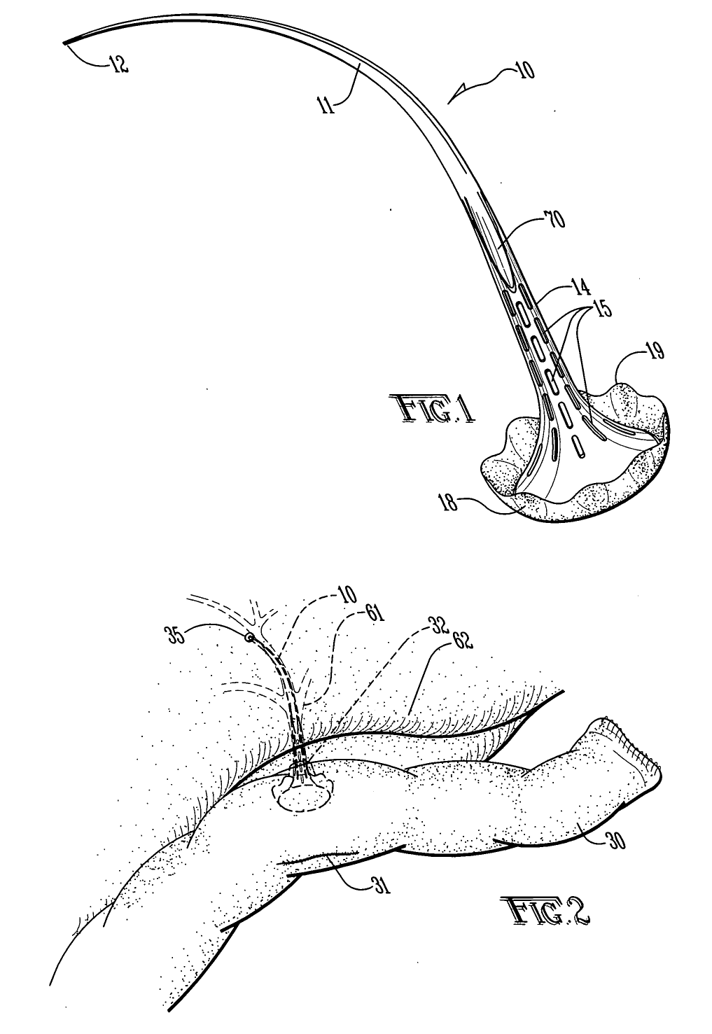

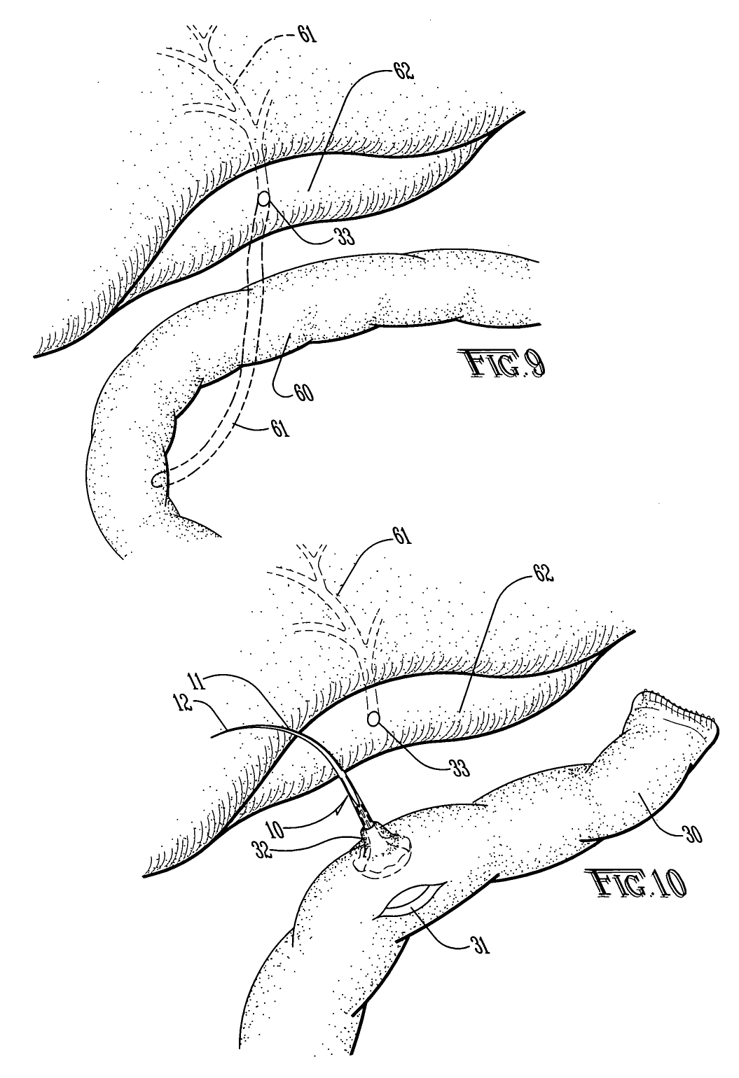

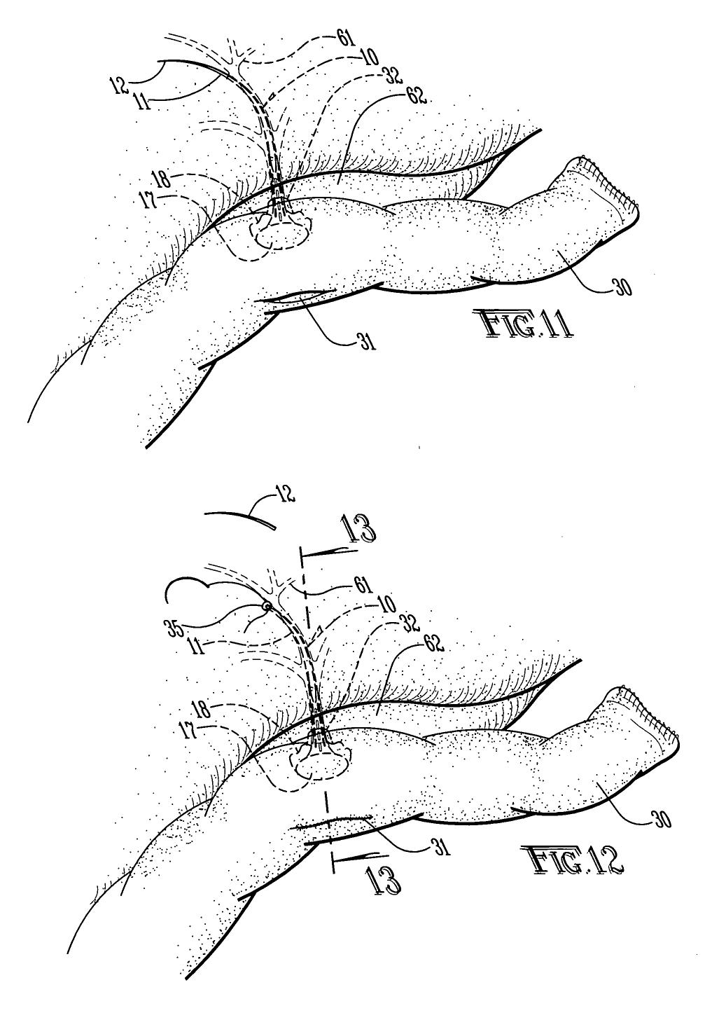

The present invention is described with reference to FIG. 1-18. The present invention relates to a catheter for creating a sutureless anastomosis. There are various medical reasons for creating an anastomosis, which is an opening or connection between two vessels or between two normally separate spaces within the human body. For example, it may be desirable to create a passage to allow the drainage of fluids from an organ whose normal outlet is blocked. One such condition involves the blockage of the bile ducts which requires the creation of a communication between the liver and the small intestine.

The preferred embodiment of the present invention as shown in FIG. 1 is a catheter 10 that includes a rigid guide portion 11 having a penetrating point 12. The guide portion 11 may be reinforced for increased rigidity. For example, as shown in FIGS. 19 and 20, a wire 13 may be embedded into the guide portion 11.

The catheter 10 also includes a fenestrated portion 14 which has at least one fenestration 15 communicating with a hollow interior 16 as shown in FIG. 8. The number of fenestrations 15 is not significant and may be adjusted to particular situations. The fenestrated portion 14 terminates in an opening 17 as shown in FIG. 13. The opening 17 has an everted rim 18. The everted rim 18 is desirably provided with a corrugated margin 19. The corrugations of the corrugated rim 19 grip the enteric mucosa as shown in FIG. 13 without compromising the blood supply.

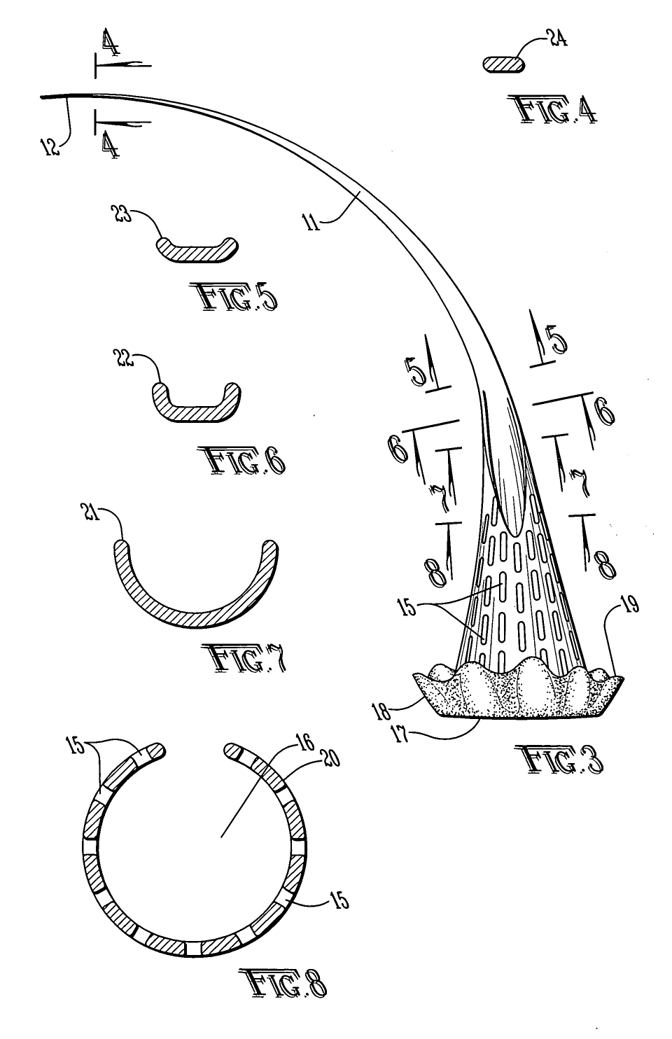

As shown in FIGS. 3-8, the guide portion 11 desirably tapers along its length from the penetrating point 12, gradually increasing in size until it smoothly merges with the fenestrated portion 14. Further, it is desirable that the cross-section of the guide portion 11 gradually opens up from an entrance 70 to the enclosed hollow interior 16 of the fenestrated portion 14 to a partially open circle 20 to a semicircular cross-section 21 to a gradually more flattened cross-sections 22, 23, 24 to the penetrating point 12. The entrance 70 provides a route from drainage from the bile duct to complete the fenestrations 15.

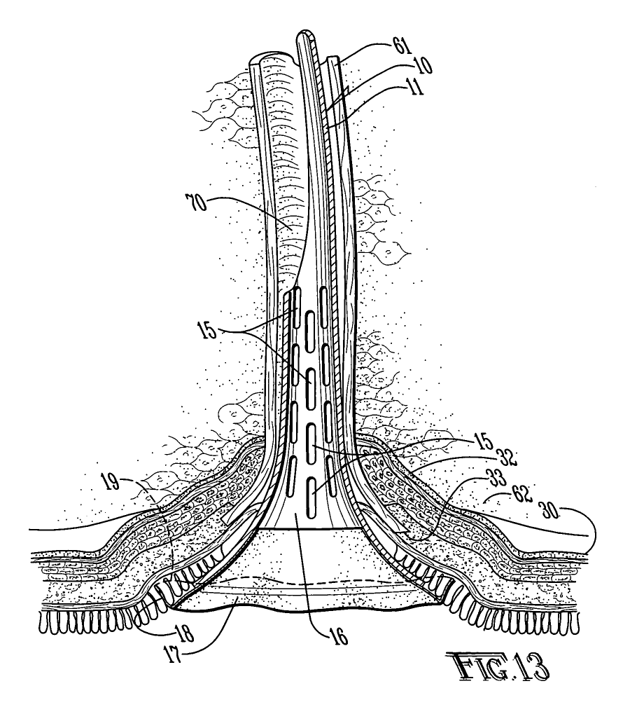

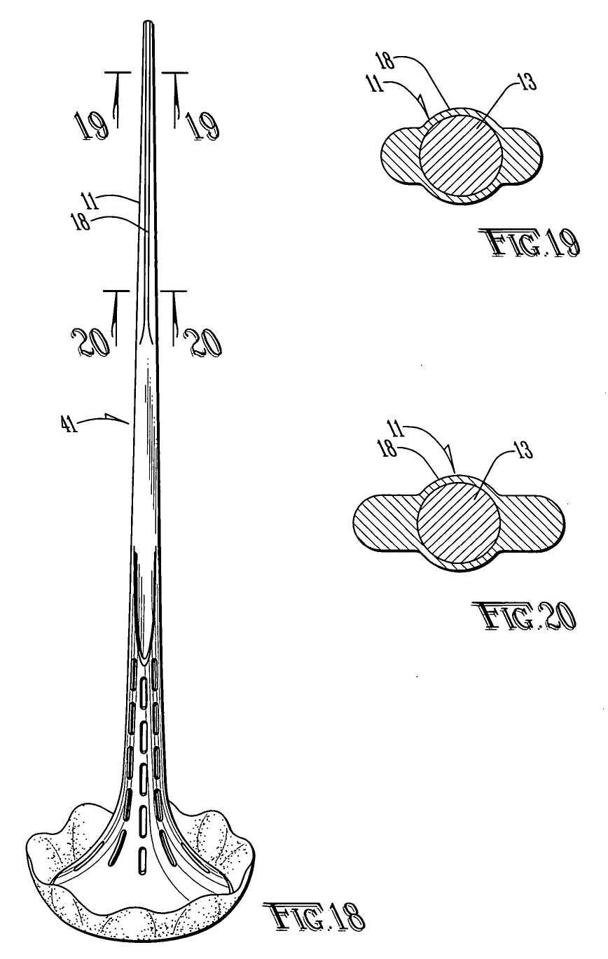

The method of use of the present invention is described with respect to FIGS. 2-13. The bile duct 61 of the liver 62 drains to the small intestine 60. If the bile duct is blocked or otherwise non-functional, an anastomosis is required. In order to create an anastomosis of the type described in Wexler and Smith, a Roux limb 30 of the small intestine 60 is created and an enterotomy 31 is made in the Roux limb 30 as shown in FIG. 10. A mucosal sleeve 32 (otherwise referred to as a graft or flap) is made as described by Wexler and Smith. The catheter 10 is inserted through the. enterotomy 31 and the guide portion 11 is passed through the mucosal sleeve 32 and into the opening 33 of the bile duct of the liver 62 as shown in FIG. 11. The rigid guide portion 11 is then treaded through the bile duct. The guide portion 11 may be hook-shaped as shown in FIGS. 1-3 and 10-12 to guide the direction of the catheter 10 or, alternatively, a metal probe may be used to guide a catheter 41 having a straight guide portion 40 as shown in FIG. 18. When resistance is felt, the penetrating point 12 of the guide portion 11 is forced through to the surface of the liver as shown in FIG. 12. The exposed guide portion 11 is then pulled tight to create the anastomosis by bringing the enteric mucosal sleeve 32 into contact with the mucosa of the bile duct. Using a plug 35, the guide portion 11 is then sutured to the liver at the surface and the exposed guide portion 11 is cut off as shown in FIG. 2. The flat shape of the guide portion 11 helps to prevent leaking at the exit site.

If the guide portion 11 is sutured to the liver with an absorbable suture, such as a polydioxanone suture (“pds”), the catheter 10 will self-dislodge into the enteric tract after the anastomosis is stable and intact. The end result is a permanent hepatico-enteric anastomosis for the drainage of bile into the lumen of the enteric tract.

The fenestrated portion 14 of the catheter 10 may be provided with varying degrees of tapering as shown in FIGS. 1, 3 and 18. The taper acts to hold the enteric mucosal sleeve 32 in place during the formation of the anastomosis. The taper also allows some accommodation to varying sizes of openings in the Roux limb 30.

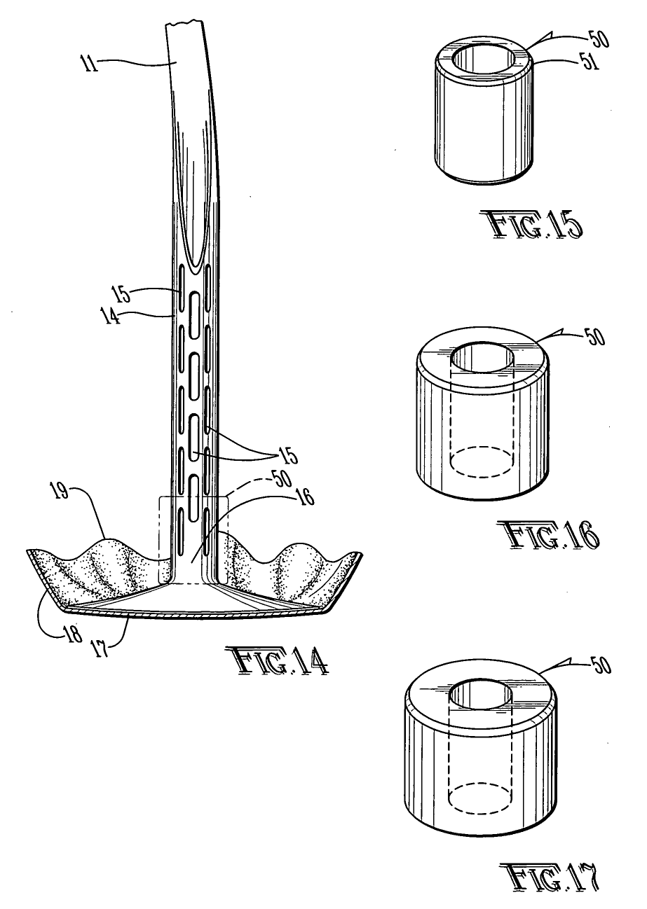

In an alternative embodiment, the fenestrated portion 14 is straight as shown in FIG. 14 without any taper. This embodiment is capable of accommodating various bile duct diameters with the use of spacers 50 with differing diameters as shown in FIGS. 15-17. The spacers 50 are placed over the catheter 10 so as to fit down against the everted rim 18. Rather than employing the taper to hold the enteric mucosa in place, the thickness of the wall of the spacer 50 defines a lip 51 at its upper end to grip the enteric mucosa and provide good contact with the mucosa of the bile duct to ensure a good anastomosis.

The present invention has been described with reference to certain preferred and alternative embodiments that are intended to be exemplary only and not limiting to the full scope of the present invention as set forth in the appended claims.

What is claimed is:

1. A fistulary catheter, comprising:
a guide portion having a first end and a second end, and a penetrating point disposed on said first end;
a fenestrated portion attached to said guide portion at said second end, said fenestrated portion having a wall enclosing a hollow interior and at least one fenestration in said wall, said wall terminating in an opening communicating with said hollow interior; and
an everted rim on said opening.

2. The fistulary catheter of claim 1, wherein said everted rim has a corrugated margin.

3. The fistulary catheter of claim 1, wherein said fenestrated portion is tapered from said everted rim toward said guide portion.

4. The fistulary catheter of claim 1, wherein said guide portion is tapered from said second end toward said first end.

5. The fistulary catheter of claim 1, wherein at least a segment of said guide portion comprises reinforcing means.

6. The fistulary catheter of claim 1, wherein said guide portion is hooked.

7. A fistulary catheter kit, comprising:
a fistulary catheter, comprising:
a guide portion having a first end and a second end, and a penetrating point disposed on said first end;
a fenestrated portion attached to said guide portion at said second end, said fenestrated portion having a wall enclosing a hollow interior and at least one fenestration in said wall, and said wall terminating in an opening communicating with said hollow interior; and
an everted rim on said opening; and
a spacer comprising a tube having a wall of a predetermined thickness enclosing an interior space and an opening at each end communicating with said interior space, said openings and said interior space configured to receive at least a part of said fenestrated portion of said fistulary catheter.

8. The fistulary catheter kit of claim 7, wherein said everted rim has a corrugated margin.

9. The fistulary catheter kit of claim 7, wherein said guide portion is tapered from said second end toward said first end.

10. The fistulary catheter kit of claim 7, wherein at least a segment of said guide portion comprises reinforcing means.

11. The fistulary catheter kit of claim 7, wherein said guide portion is hooked.
